(19) United States
(12) Patent Application Publication
(10) Pub. No.: US 2015/0072967 A1
(43) Pub. Date: Mar. 12, 2015

(54) TAUROURSODEOXYCHOLIC ACID ATTENTUATES OR ABOLISHES FORMATION AND DEPOSITION OF AMYLOID-B PEPTIDE

(71) Applicant: Metselex, Inc., Minneapolis, MN (US)

(72) Inventors: Cecilia M.P. Rodrigues, Lisbon (PT); Rudi D'Hooge, Heverlee-Leuven (BE)

(73) Assignee: Metselex, Inc., Minneapolis, MN (US)

(21) Appl. No.: 14/386,640

(22) PCT Filed: Mar. 19, 2013
(86) PCT No.: PCT/US2013/032960

§ 371 Date: Sep. 19, 2014

Related U.S. Application Data
(60) Provisional application No. 61/613,117, filed on Mar. 20, 2012.

(51) Int. Cl. A61K 31/575

(52) U.S. Cl. CPC A61K 31/575
USPC 514/182

(57) ABSTRACT

Methods of preventing or retarding or reversing or abolishing the onset and preventing the onset of neurodegenerative disease are discussed. This is achieved through the administration of a bile acid, a salt of the bile acid, an analog of the bile acid or any combinations of these compounds. The bile acid abolishes or interferes or down-regulates metabolic pathways leading to the onset of neurodegenerative diseases. The bile acid also activates metabolic pathways leading to the slowing or reversing or complete abolishment of the progression of neurodegenerative disease.

### BACKGROUND OF THE INVENTION

### Alzheimer's Disease

Alzheimer's disease (AD) is the most prevalent form of dementia, resulting in progressive neuronal death and debilitating damage to brain loci that mediate memory and higher cognitive function. Accumulation of amyloid-β (Aβ) peptide has been shown to play a critical role in the pathogenesis of AD. Among its two predominant forms, Aβ₁₋₄₂ possesses stronger aggregation and deposition propensity than Aβ₁₋₄₀. Accumulation of Aβ in the brain is associated with mutations in amyloid precursor protein (APP), presenilin 1 (PS1), and presenilin 2 (PS2) genes. Aβ peptides are generated by successive proteolysis of APP, a large transmembrane glycoprotein that is initially cleaved by β-secretase, and subsequently by γ-secretase in the transmembrane domain. The γ-secretase complex consists of presenilin and at least three other integral membrane proteins. Usually, γ-secretase does not proteolyse the full-length proteins, but so-called COOH-terminal fragments (CTFs), produced from the full-length proteins by another protease. While pathogenic genetic mutations have been implicated in ˜2% of AD cases, the proximal events that underlie the common, sporadic form of the disease are incompletely understood.

Over the past decades, the molecules that control APP processing into Aβ have been the focus of intense investigation. In this respect, connective tissue growth factor (CTGF) has been recently implicated in AD pathogenesis by increasing Aβ peptides steady-state levels, possibly through a mechanism that involves γ-secretase activation. Much like APOE, A2M and SORLA, CTGF is a ligand for the low density lipoprotein receptor-related protein, and its expression correlates with the progression of AD clinical dementia and amyloid neuritic plaque neuropathology.

### SUMMARY OF THE INVENTION

The present invention provides a method of ameliorating learning and memory deficits through the administration of tauroursodeoxycholic acid (TUDCA), which reduces the accumulation of Aβ deposits in the brain. The patient is a human patient, while the administering step involves administering, through various means, an amount of TUDCA, in any formulation in any combination that is effective in providing the necessary pharmacological benefit.

One feature of the present invention involves the administering of an effective amount of TUDCA or any of its analogs or formulations or any combination thereof. The mode of administering TUDCA includes, but is not limited to, intravenously, parenterally, orally or intramuscularly or any combination of these methods.

Alzheimer's disease is a neurodegenerative disorder which is typically characterized by cognitive deficit. For example, Alzheimer's disease could include memory loss and learning impairment.

Herein, a “patient” includes a human or any mammal.

### BRIEF DESCRIPTION OF THE FIGURES

FIG. 1 depicts reduction in levels of amyloid deposits in TUDCA-treated APP/PS1 mice.

FIG. 2 depicts decreased Aβ deposits in the brains of APP/PS1 mice compared with untreated APP/PS1 mice due to TUDCA treatment.

FIG. 3 shows decreased astrocytic activation in brains of APP/PS1 mice due to TUDCA treatment.

FIG. 4 depicts decreases in microglial activation in the brains of APP/PS1 mice due to TUDCA treatment.

FIG. 5 depicts the prevention of neuronal integrity loss in the brains of APP/PS1 mice.

FIG. 6 shows that TUDCA regulates the expression of lipid-metabolism mediators associated with AD pathology in the brains of APP/PS1 mice.

FIG. 7 shows that TUDCA modulates protein levels of lipid-metabolism mediators associated with AD pathology in the brains of APP/PS1 mice.

FIG. 8 shows that TUDCA treatment decreases the production of APP-CTFs and Aβ in the brains of APP/PS1 mice.

FIG. 9 shows that TUDCA decreases Aβ₁₋₄₀ and Aβ₁₋₄₂ levels in the brains of APP/PS1 mice.

### DETAILED DESCRIPTION OF THE INVENTION

The current invention details a method for treating a patient exhibiting learning and memory deficits attributed to, among other disorders, Alzheimer's disease. In the existing state of medical arts there is no effective therapy that would ameliorate or reverse or prevent Alzheimer's disease. Treatment here is defined by either preventing or slowing the onset of Alzheimer's, or any neurodegenerative disease, or slowing down the progression of the disease.

Alzheimer's disease is a disorder that results in progressive neuronal death and debilitating damage to brain loci that mediate memory and higher cognitive function. Progression of Alzheimer's disease is characterized by the accumulation of amyloid-β (Aβ).

Treatment outlined in the current invention is enabled by a bile acid. Examples of a bile acid are TUDCA, UDCA, or any analogs, derivatives, precursors thereof.

The endogenous bile acid tauroursodeoxycholic acid (TUDCA) is a strong neuroprotective agent in several experimental models of disease, including neuronal exposure to Aβ. TUDCA is a potent anti-apoptotic agent in neuronal cells exposed to Aβ and a powerful neuroprotective strategy in animal models of neuronal degeneration. TUDCA specifically modulates Aβ-induced toxicity by inhibiting organelle-driven apoptosis and interfering with upstream molecular targets of p53 pathways, although without changing secondary structures and fibrillogenic propensities of Aβ peptides in vitro. The molecular mechanisms underlying TUDCA neuroprotective properties appear to be complex and may engage a number of different molecular targets, possibly involving gene regulation. Nevertheless, TUDCA strongly downregulates by greater than 10-fold A2M and CTGF expression, as demonstrated by DNA microarray gene expression analysis of hepatocytes. TUDCA can not only inhibit pathways that would lead to the onset of neurodegenerative disorders, but also activate pathways that either inhibit the onset or slow the progression of neurodegenerative disorders. In addition to Alzheimer's disease, bile acids are effective in treating other neurodegenerative disorders such as Parkinson's, Huntington's, etc. in part because they exhibit similar characteristics of tissue degeneration, in additional to reducing reactive oxygen species.

TUDCA is an orally bioavailable and central nervous system penetrating agent, known to cross the blood-brain barrier. Given the effect of TUDCA in regulating lipid metabolism gene expression and the role of lipid mediators in modulating Aβ metabolism, the inventors believe that TUDCA reduces Aβ toxicity by interfering with its production and accumulation.

The amelioration of Alzheimer's disease systems by the administration of TUDCA was accompanied by reduced glial activation and neuronal integrity loss in TUDCA-fed APP/PS1 mice compared to untreated APP/PS1 mice. Furthermore, the inventors found that TUDCA regulated lipid-metabolism mediators involved in Aβ production and accumulation in the brains of transgenic mice. Overall amyloidogenic APP processing was reduced with TUDCA treatment, in association with, but not limited to, modulation of γ-secretase activity. Consequently, a significant decrease in Aβ₁₋₄₀ and Aβ₁₋₄₂ levels was observed by the inventors in both hippocampus and frontal cortex of TUDCA-treated APP/PS1 mice, suggesting that chronic feeding of TUDCA interferes with Aβ production, possibly through the regulation of lipid-metabolism mediators associated with APP processing.

Even though TUDCA is known to prevent cell death by apoptosis, its inhibition of production and accumulation of Aβ associated with Alzheimer's disease was unexpected. At the time of the investigation, it was not known if the mechanism underlying prevention of apoptosis by TUDCA was linked to any pathways effecting the production and accumulation of Aβ presumably due to the downregulation of lipid metabolism

The methods of the current invention are associated with the utilization of a hydrophilic bile acid, its salts thereof and analogs thereof, and combinations thereof. These bile acids are more hydrophilic than its isomer chenodeoxycholic acid (CDCA). The hydrophilic bile acids also include ursodeoxycholic acid (UDCA).

Analogs of TUDCA include, among others, conjugated derivatives of bile acids such as nor-ursodeoxycholic acid, glycol-ursodeoxycholic acid, ursodeoxycholic acid 3-sulfate, ursodeoxycholic acid 7-sulfate, and ursodeoxycholic acid 3,7-sulfate.

Amelioration of symptoms of Alzheimer's disease by hydrophilic bile acids can be evaluated by investigating the ability of these bile acids to inhibit the formation and accumulation of Aβ in the brain or anywhere in the body.

These hydrophilic bile acids are used in amounts ranging from 1.0-60 mg/kg body weight to treat Alzheimer's or other neurodegenerative disease by either or both prophylactic or therapeutic treatments. Treatment involves prevention of onset or retardation or complete reversal of any or all symptoms or pharmacological or physiological or neurological or biochemical indications associated with Alzheimer's disease. Treatment can begin wither with the earliest detectable symptoms or established symptoms of Alzheimer's disease.

The “effective” amounts range from 1.0-60.0 mg/kg body weight. This dosage will prevent or retard or completely abolish any or all pathophysiological features associated with various stages (late or end) Alzheimer's disease (sporadic or familial). In a further embodiment, effective amounts range from 5.0-45.0 mg/kg body weight. In yet a further embodiment, effective amounts range from 15.0-40.0 mg/kg body weight. In yet another embodiment, effective amounts range from 25.0-35.0 mg/kg body weight.

The hydrophilic bile acids can be combined with a formulation that includes a suitable carrier. Preferably, the compounds utilized in the formulation are of pharmaceutical grade. This formulation can be administered to the patent, which includes any mammal, in various ways which are, but not limited to, oral, intravenous, intramuscular, nasal, or parenteral (including, and not limited to, subcutaneous, intramuscular, intraperitoneal, intravenous, intrathecal, intraventricular, direct injection into the brain or spinal tissue).

Formulations presented to the patient may be prepared by any of the methods in the realm of the art of pharmacy. These formulations are prepared by mixing the biologically-active hydrophilic bile acid into association with compounds that comprise a carrier. The carrier can be liquid, granulate, solid (coarse or finely broken), liposomes (including liposomes prepared in combination with any non-lipid small or large molecule), or any combination thereof.

The formulation in the current invention can be furnished in distinct units including, but not limited to, tablets, capsules, caplets, lozenges, wafers, troches with each unit containing specific amounts of the active molecule for treating Alzheimer's disease. The active molecule can be incorporated either in a powder, encapsulated in liposomes, in granular form, in a solution, in a suspension, in a syrup, in any emulsified form, in a drought or in an elixir.

Tablets, capsules, caplets, pills, troches, etc. that contain the biologically-active hydrophilic bile acid can contain binder (including, but not limited to, corn starch, gelatin, acacia, gum tragacanth), an excipient agent (including but not limited to dicalcium phosphate), a disintegrating agent (including but not limited to corn starch, potato starch, alginic acid), a lubricant (including but not limited to magnesium stearate), a sweetening agent (including but not limited to sucrose, fructose, lactose, aspartame), a natural or artificial flavoring agent. A capsule may additionally contain a liquid carrier. Formulations can be of quick or sustained or extended-release type.

Syrups or elixirs can contain one or several sweetening agents, preservatives, crystallization-retarding agents, solubility-enhancing agents, etc.

Any or all formulations containing the biologically-active hydrophilic bile acids can be included into the food (liquid or solid or any combination thereof) of the patient. This inclusion can either be an additive or supplement or similar or a combination thereof.

Parenteral formulations are sterile preparations of the desired biologically-active hydrophilic bile acid and can be aqueous solutions, dispersions of sterile powders, etc., that are isotonic with the blood physiology of the patient. Examples of isotonic agents include, but are not limited to, sugars, buffers (e.g., saline), or any salts.

Formulations for nasal spray are sterile aqueous solutions containing the biologically-active hydrophilic bile acid along with preservatives and isotonic agents. The sterile formulations are compatible with the nasal mucous membranes.

The formulation can also include a dermal patch containing the appropriate sterile formulation with the active agent. The formulation would release the active agent into the blood stream either in sustained or extended or accelerated or decelerated manner.

The formulation can also consist of a combination of compounds, in any of the aforementioned formulations designed to traverse the blood-brain barrier.

### EXAMPLES

In the following examples, the function of TUDCA in its various forms in arresting or delaying or entirely preventing the onset of Alzheimer's disease is further characterized. Specifically, TUDCA treatment led to the prevention or reduction (partial or complete) of the formation or accumulation of amyloid-β (Aβ) or any other type of plaque in the brain or any other part of the entire body. TUDCA treatment regulated all aspects of lipid metabolism involved in the formation or clearance or internalization of Aβ. TUDCA treatment can improve any or all aspects of recognition and spatial and contextual memory. Therefore, it can be deduced that TUDCA is unique in its pharmacological and physiological actions in the treatment of Alzheimer's disease.

### Methods

Transgenic mice and TUDCA treatment. APP/PS1 double-transgenic mice express human APP containing the KM670/671NL Swedish double mutation and human PS 1 carrying the L166P mutation under the control of a neuron-specific murine Thy-1 minigene promoter. The two transgenes cosegregate in these mice. APP/PS1 mice were maintained on a C57BL/6J genetic background and genotyped by PCR analysis of genomic DNA from tail biopsies. All animals were kept in standard animal cages under conventional laboratory conditions (12 h light/dark cycle, 22° C.), with ad libitum access to food and water. The animals were randomized for therapy trials and coded, and the operators remained double blinded to which treatment they received, until the completion of data collection. Male APP/PS1 transgenic mice and wild-type littermates were randomly assigned into four groups: TUDCA-treated and untreated (control) APP/PS1 mice and TUDCA-treated and untreated (control) wild-type mice. Animals were fed a diet of standard laboratory chow supplemented with either 0.4% (wt/wt) TUDCA (sodium salt; Prodotti Chimici e Alimentari S.p.A., Basaluzzo, Italy), or no bile acid, custom made by Harlan (Harlan Laboratories Models, S.L., Barcelona, Spain). Treatment was started when the mice were 2 months old and continued for 6 months. The dose and duration of TUDCA, whose pharmacokinetics has been extensively studied in rodents, were chosen based on pilot studies using APP/PS1 mice and other animal studies. The use of 2-month-old APP/PS1 mice to receive TUDCA treatment was based on previous reports demonstrating that amyloid deposits in this model start at ˜3 months and behavioral deficits are absent until 7-8 months.

Immunohistochemistry. After TUDCA treatment for 6 months, animals were anesthetized with an intraperitoneal injection of ketamine (0.05 mg/kg) and perfused first with PBS and then with 4% paraformaldehyde in PBS. One hemisphere of the brain was snap-frozen for protein and RNA extraction. The other hemisphere was dehydrated, treated with xylene, and embedded in paraffin. Serial 4 μm-thick coronal brain sections were cut on a microtome, and mounted on SuperFrost-Plus (Thermo Scientific, Rockford, Ill., USA) glass slides. Paraffin-embedded brain sections were deparaffined, rehydrated and boiled 3 times in 10 mM citrate buffer, pH 6. Sections were then incubated for 60 min in blocking buffer [10% (v/v) normal donkey serum (Jackson ImmunoResearch Laboratories Inc., West Grove, Pa., USA) in PBS containing 0.1% (v/v) Triton X-100 (Sigma-Aldrich, St. Louis, Mo., USA)] and subsequently in appropriately diluted primary antibodies overnight at 4° C. After rinsing, the primary antibody was developed by incubating with cyanine 2 (Cy2, Jackson)- or Alexa Fluor 594 (Invitrogen, Grand Island, N.Y., USA)-conjugated secondary antibodies against the corresponding species, for 1 h at room temperature. The following primary antibodies were used for immunohistochemistry: Aβ plaques were immunostained with a mouse monoclonal anti-Aβ (6E10; Signet Laboratories Inc., Dedham, Mass., USA; 1:1000); astrocytes were stained with a mouse monoclonal glial fibrillary acidic protein (GFAP) antibody (GA5; Millipore Corporation, Temecula, Calif., USA; 1:400); microglia were stained with a rabbit polyclonal Iba-I antibody (Wako Pure Chemicals, Richmond, Va., USA; 1:100); neuronal cell bodies and dendrites were labeled with a rabbit polyclonal microtubule associated protein 2 (MAP2) antibody (Millipore; 1:100). On control sections, where primary antibody was replaced by blocking buffer, no staining was observed.

Histochemistry. Paraffin-embedded brain sections were deparaffined, rehydrated and stained with Thioflavin T (Sigma), a general marker of amyloid deposits. Incubation with freshly filtered 0.05% Thioflavin T solution in PBS was performed for 8 min at room temperature.

Image analysis and semiquantification of immunofluorescence. Images were acquired with an Axioskop fluorescence microscope (Carl Zeiss GmbH, Hamburg, Germany). Semiquantitative analysis of mean fluorescence intensities (MFIs) of GFAP, Iba-I, and MAP2 were performed using NIH Image J software. Eight images of slides were obtained per hippocampal and cortical regions. Images were converted into a 8-bit format, and the background was subtracted. An intensity threshold was set and kept constant for all images analyzed. MFI per square millimeter area was calculated by dividing the MFI units by the area of outlined regions and is presented as a bar graph. The total number of thioflavin- and Aβ-positive plaques in the hippocampus and frontal cortex was counted and presented as plaque number per square millimeter.

Real-time PCR. Total RNA from dissected hippocampus and frontal cortex was isolated with TRIzol (Invitrogen), according to the manufacturer's instructions. Samples were homogenized in TRIzol using a motor-driven Bio-vortexer (No 1083; Biospec Products, Bartlesfield, Okla.) and disposable RNAse/DNAse free sterile pestles (Thermo Scientific). RNA was quantified using a NanoDrop spectrophotometer, and typically showed A260/280 ratios between 1.9 and 2.1. cDNA was made using Superscript II Reverse Transcriptase (Invitrogen) according to the manufacturer's instructions. Real-time quantitative PCR analysis was performed in the 7300 Real-Time PCR System (Applied Biosystems, Foster City, Calif., USA), using SYBR Green PCR master mix (Fermentas International Inc., Glen Burnie, Md., USA). Triplicate reactions were run per sample. The expression levels relative to GAPDH were calculated using the ΔΔCₜ method. TUDCA-untreated wild-type mice were used as the calibrator. The n-fold change in expression was obtained using the formula: 2^{-ΔΔCt}. Primer sequences are presented in Table 1.

Western blot analysis. Total protein extracts from dissected hippocampus and frontal cortex were prepared in lysis buffer, following standard protocols. Protein concentrations were determined using the Bio-Rad protein assay kit, according to the manufacturer's specifications. Sixty μg of total protein extracts were separated on 6 and 12% sodium dodecyl sulphate-polyacrylamide gel electrophoresis. After electrophoretic transfer onto nitrocellulose membranes, and blocking with a 5% milk solution, membranes were incubated overnight at 4° C. with the following primary antibodies: rabbit polyclonal antibody reactive to CTGF (Abcam, Cambridge, UK), rabbit polyclonal antibody reactive to APOE (Santa Cruz Biotechnology, Santa Cruz, Calif., USA), rabbit polyclonal antibody reactive to SORLA (Santa Cruz), rabbit polyclonal antibody reactive to A2M (Santa Cruz). Finally, secondary goat anti-mouse or anti-rabbit IgG antibody conjugated with horseradish peroxidase (BioRad Laboratories, Hercules, Calif., USA) was added for 3 h at room temperature. The membranes were processed for protein detection using the SuperSignal substrate (Pierce Biotechnology, Rockford, Ill., USA). β-Actin (AC-15; Sigma-Aldrich) was used as loading control. The relative intensities of protein bands were analyzed using the QuantityOne Version 4.6 densitometric analysis program (Bio-Rad) and normalized to the respective loading control.

γ-secretase activity. Assessment of γ-secretase activity was performed by detection of the ˜7 KDa carboxyl terminal fragment (CTF)-γ cleavage product of APP. Briefly, 60 μg of total protein extracts were resolved electrophoretically in 10-20% Tris-Tricine gels (BioRad), and CTF-γ (and CTF-β) identified using an anti-APP, C-terminal rabbit polyclonal antibody (Sigma-Aldrich; 1:2000). After stripping, membranes were incubated with the primary mouse monoclonal antibody reactive to Aβ (6E10; Signet; 1:1000) to detect total Aβ peptide. Immunoreactivities were visualized as described above.

Sandwich ELISA. Total protein extracts from dissected hippocampus and frontal cortex were used to determine Aβ concentration. Total Aβ₁₋₄₀ or Aβ₁₋₄₂ content was measured by sandwich ELISA (Millipore) according to the manufacturer's instructions. Values were normalized to the respective total protein concentration and expressed as % of wild-type control.

Statistical analysis. Data were analyzed by the one-way analysis of variance (ANOVA), and differences between groups were determined by post hoc Bonferroni's test (Prism 2.01; GraphPad Software, Inc, San Diego, Calif., USA). Comparison of data from two groups was made by Student's two-tailed unpaired t test. Values of p<0.05 were considered significant.

### Results

### TUDCA Prevents Aβ Plaque Accumulation in APP/PS1 Mice

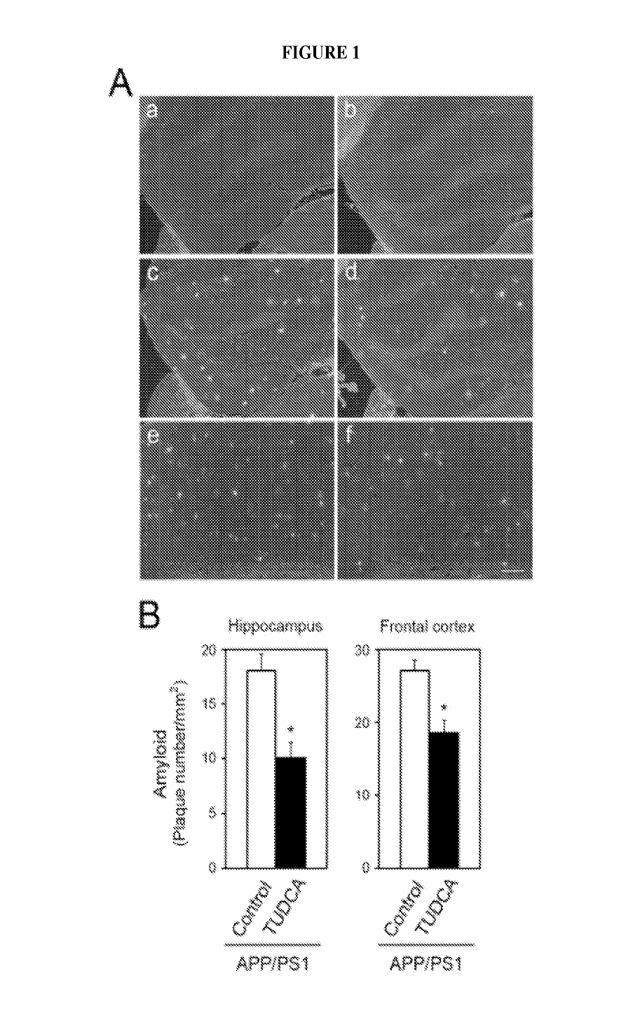

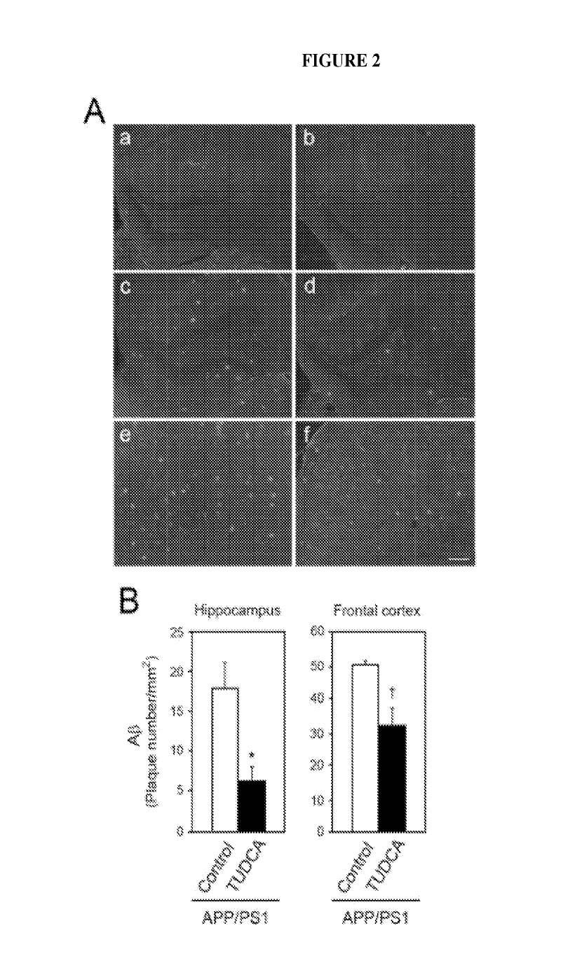

Mutations of APP and PS 1, as observed in APP/PS1 double transgenic mice lead to an increase in Aβ production and deposition. In this study, we investigated the effect of TUDCA on Aβ deposition in the brains of wild-type and APP/PS1 mice. A general histological evaluation of amyloid plaque burden was performed with thioflavin. Thioflavin staining demonstrated a clear abundance of amyloid plaques in the brains of control transgenic mice, whereas deposits were absent in both control and TUDCA-treated wild-type mice (FIG. 1A). Importantly, a marked reduction of thioflavin plaque number was observed in hippocampus and frontal cortex of transgenic mice treated with TUDCA compared to control transgenic mice (p<0.01) (FIGS. 1A and B). Aβ immunohistochemistry confirmed the presence of Aβ deposits in APP/PS1 transgenic mice, but not in wild-type mice (FIG. 2A). Furthermore, Aβ plaque number was strongly decreased by almost 65% in the hippocampus and 40% in the frontal cortex of TUDCA-treated APP/PS1 mice compared with untreated transgenic mice (p<0.01 and p<0.05, respectively) (FIGS. 2A and B). These results demonstrate that TUDCA treatment significantly attenuates Aβ levels in both the hippocampus and frontal cortex of APP/PS1 transgenic mice, suggesting an inhibitory effect of TUDCA on Aβ deposition. Importantly, TUDCA improved recognition, and spatial and contextual fear memory in transgenic mice compared to control transgenic mice.

### TUDCA Inhibits Activation of Astrocytes and Microglia in APP/PS1 Mice

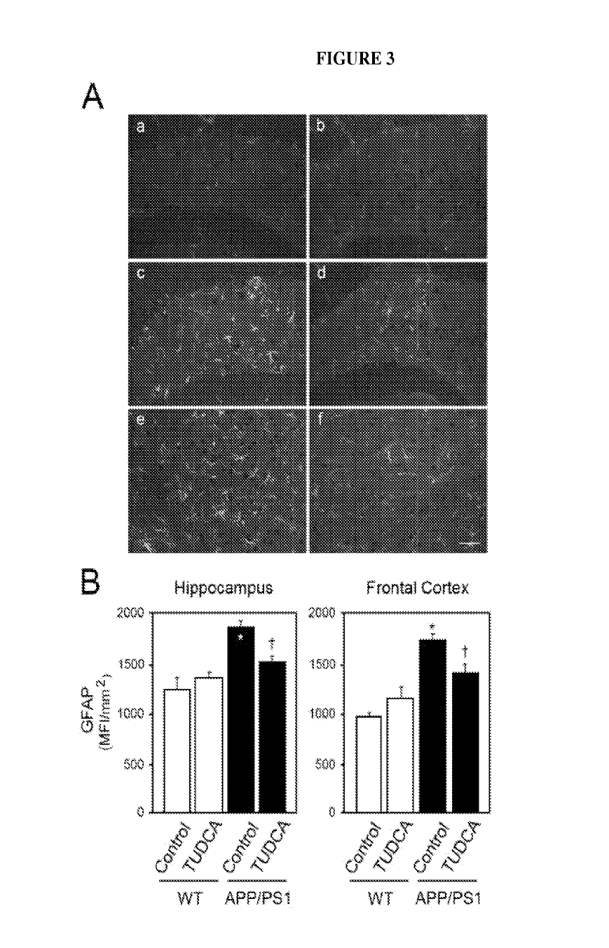

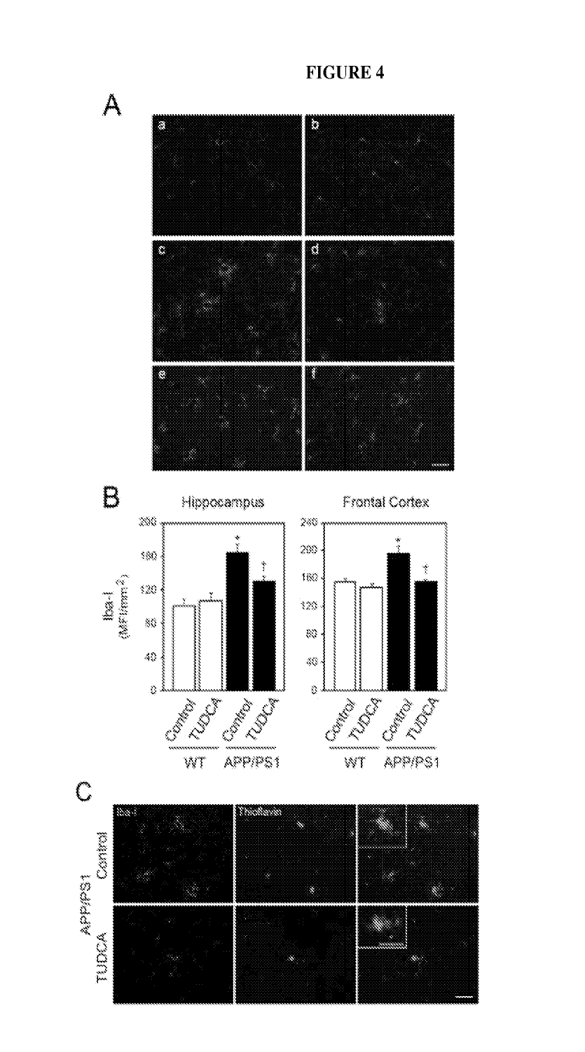

In the brains of human AD patients and transgenic AD mouse models, infiltration of activated astrocytes and microglia is observed in the area of Aβ plaques, contributing to an inflammatory process that develops around injury in the brain. To investigate whether TUDCA prevents this potentially neurotoxic, inflammatory response, we compared astrocytic and microglial reactivity in TUDCA-treated and untreated APP/PS1 and wild-type mice. Activated astrocytes were visualized by GFAP immunofluorescence. GFAP staining demonstrated a marked increase of reactive astrocytes in the brains of control transgenic mice compared to wild-type mice (p<0.01) (FIGS. 3A and B). In contrast, GFAP immunoreactivity was decreased in both hippocampus and frontal cortex of TUDCA-treated APP/PS1 mice (p<0.05) (FIGS. 3A and B). Activated microglia were visualized by Iba-I immunostaining. As shown in FIG. 4, a significant elevation of Iba-I immunoreactivity was observed in control APP/PS1 mice compared to wild-type mice (p<0.01). Noteworthy, significantly less Iba-I immunoreactivity was observed in TUDCA-treated APP/PS1 mice relative to control APP/PS1 mice (p<0.05) (FIGS. 4A and B). To visualize the reactive microglia surrounding amyloid plaques, sections were stained with thioflavin after Iba-I immunofluorescence. Double-staining of Iba-I and amyloid plaques showed accumulation of reactive microglia around the plaques in the brains of control APP/PS1 mice, whereas fewer reactive microglia were observed around the less amyloid plaques in the brains of TUDCA-treated APP/PS1 mice (FIG. 4C). These results suggest that inflammation is modulated by TUDCA in APP/PS1 mice.

### TUDCA Prevents Loss of Neuronal Integrity in APP/PS1 Mice

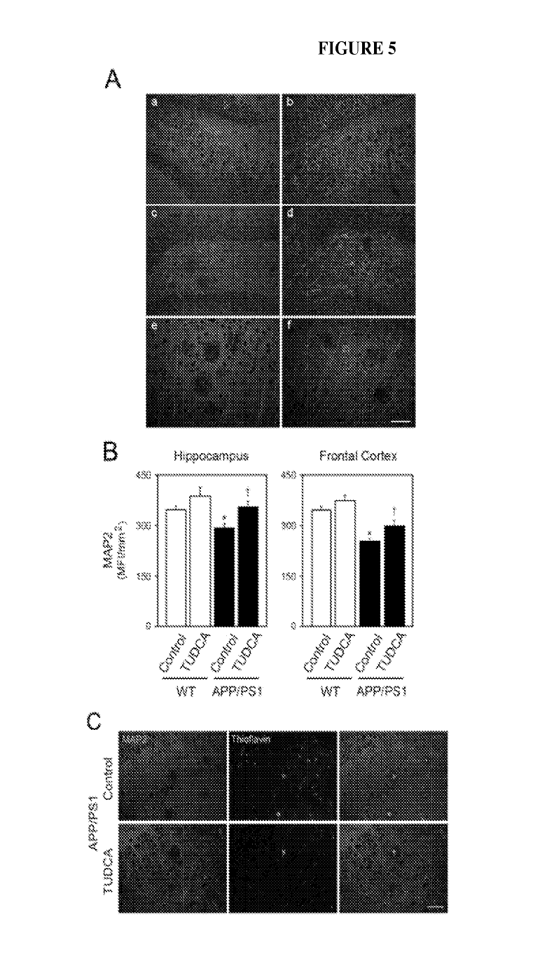

Neuronal degeneration and loss observed in the brains of AD patients and in the brains of APP/PS1 transgenic mice is hypothesized to be exacerbated by an inflammatory reaction. Given the inhibitory effect of TUDCA on glial activation, we examined the levels of the neuronal marker MAP2, expressed on neuronal cell bodies and dendrites, in the brains of wild-type and APP/PS1 mice treated or untreated with TUDCA. Consistent with previous reports showing decreased MAP2 immunoreactivity in the hippocampus of APP/PS1 mice, our results showed that control APP/PS1 mice displayed significantly less brain MAP2 immunoreactivity when compared to wild-type mice (p<0.01) (FIGS. 5A and B). Importantly, TUDCA treatment prevented the loss of MAP2 immunoreactivity in both the hippocampus and frontal cortex of APP/PS1 mice (p<0.05) (FIGS. 5A and B). Double staining of MAP2 and amyloid plaques showed a considerable degeneration of neurons, characterized by damage or loss of neuronal fibers surrounding amyloid plaques in the brains of control APP/PS1 mice (FIG. 5C). In contrast, a significant improvement in the integrity of the neuronal fibers was observed around the less amyloid plaques in the brains of TUDCA-treated APP/PS1 mice. These data suggest that TUDCA treatment decreases the rate of neuronal degeneration in APP/PS1 mice.

### TUDCA Modulates Lipid Metabolism Mediators in APP/PS1 Mice

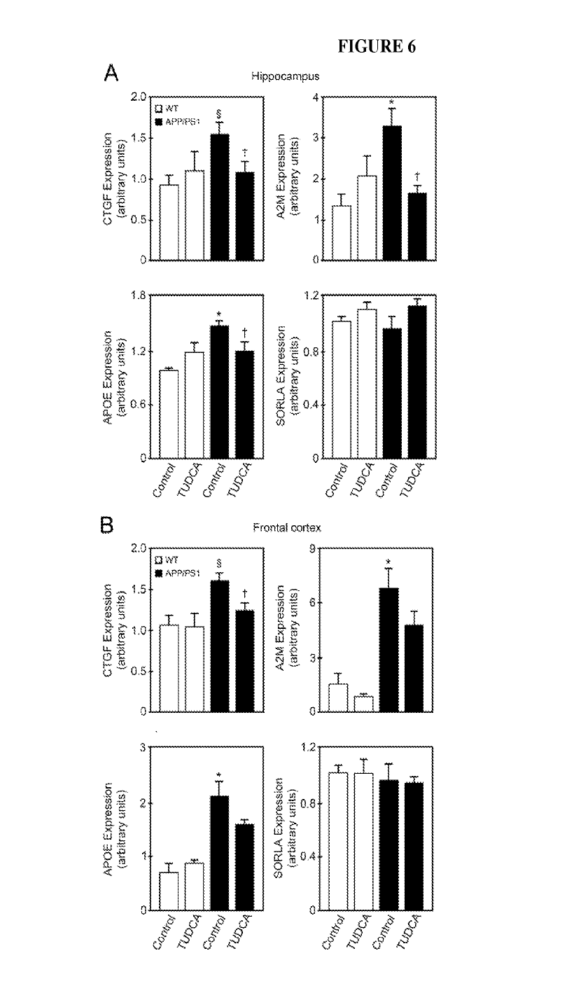

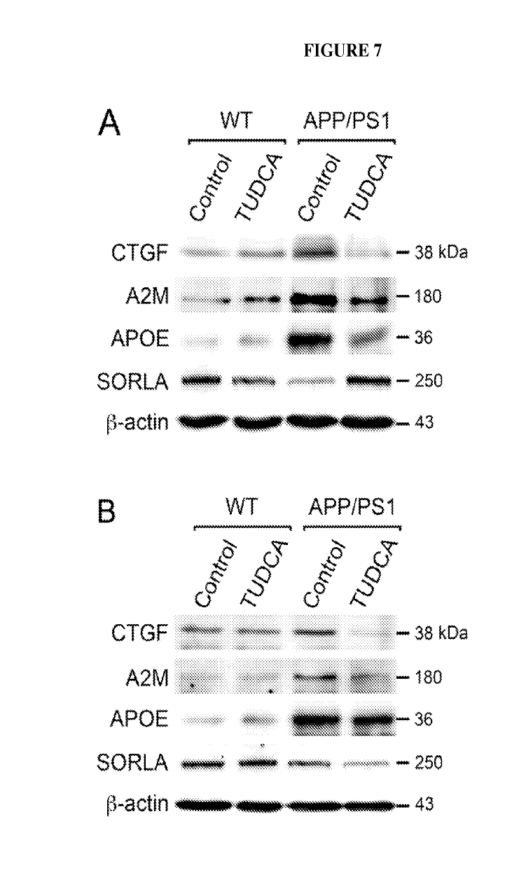

Given the effect of TUDCA in regulating the expression of lipid-metabolism mediators in hepatocytes, and the role of lipid mediators in modulating Aβ metabolism, we assessed CTGF, A2M, APOE, and SORLA levels in the brains of wild-type and APP/PS1 mice treated and untreated with TUDCA. Quantitative real-time PCR analysis demonstrated increased expression of CTGF, A2M, and APOE in the hippocampus and frontal cortex of control APP/PS1 mice compared to wild-type mice (p<0.05 and p<0.01), whereas a trend for decreased expression of SORLA in APP/PS1 mice was observed in both cerebral regions (FIGS. 6A and B). In agreement with the microarray gene expression profile in hepatocytes and a potential neuroprotective mechanism for TUDCA, this bile acid significantly decreased CTGF, A2M, and APOE expression in APP/PS1 mice hippocampus (p<0.05), while slightly increasing SORLA expression (FIG. 6A). In the frontal cortex, only CTGF expression was significantly decreased by TUDCA treatment of APP/PS1 mice (p<0.05), although a trend for decreased A2M and APOE expression was also observed (FIG. 6B). Similar results were obtained at the protein level by Western blot analysis of total protein extracts from hippocampus (FIG. 7A) and frontal cortex (FIG. 7B). These results indicate that TUDCA regulates the levels of lipid-metabolism mediators involved in Aβ production and accumulation.

### TUDCA Prevents APP Processing and Aβ Production in APP/PS1 Mice

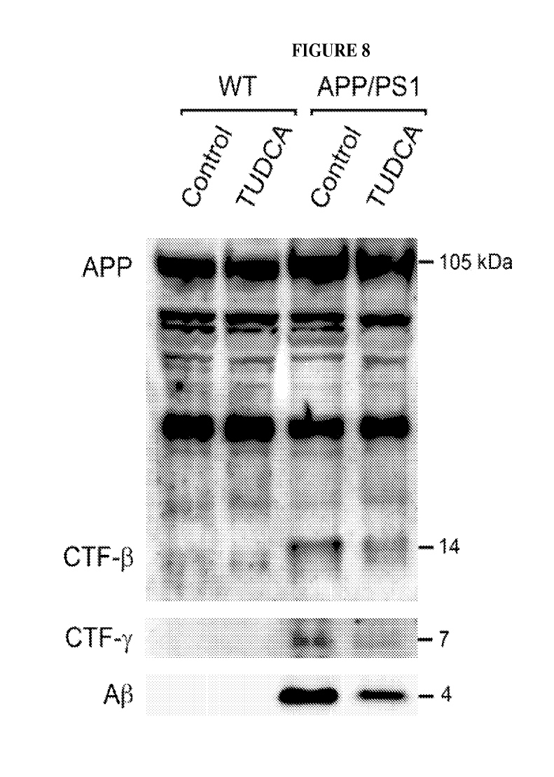

Having established the ability of TUDCA in modulating brain lipid-metabolism mediators involved in Aβ metabolism, we hypothesized that decreased amyloidogenic processing of APP is the mechanism underlying the diminished amyloid pathology in TUDCA-fed APP/PS1 mice. To determine whether TUDCA influences γ-secretase activity, brain tissues from untreated and TUDCA-treated APP/PS1 mice or wild-type littermates were subjected to electrophoresis in 10-20% Tris-Tricine gels, followed by immunoblot analysis of APP-CTF-γ using an anti-APP-CTF antibody, which also recognizes full-length APP. A decrease in the production of CTF-γ was observed in TUDCA-treated APP/PS1 mice compared to control transgenic mice, whereas full-length APP levels remained unchanged (FIG. 8). Curiously, CTF-β levels were also found decreased in TUDCA-fed transgenic mice compared to control transgenic mice (FIG. 8). In agreement with the decreased levels of CTF-γ and -β, total Aβ levels were also decreased in TUDCA-treated APP/PS1 mice compared to control APP/PS1 mice (FIG. 8). These findings suggest that TUDCA influences APP processing via modulation of γ-secretase activity. Moreover, these results point toward a reduction in overall amyloidogenic APP processing with TUDCA treatment.

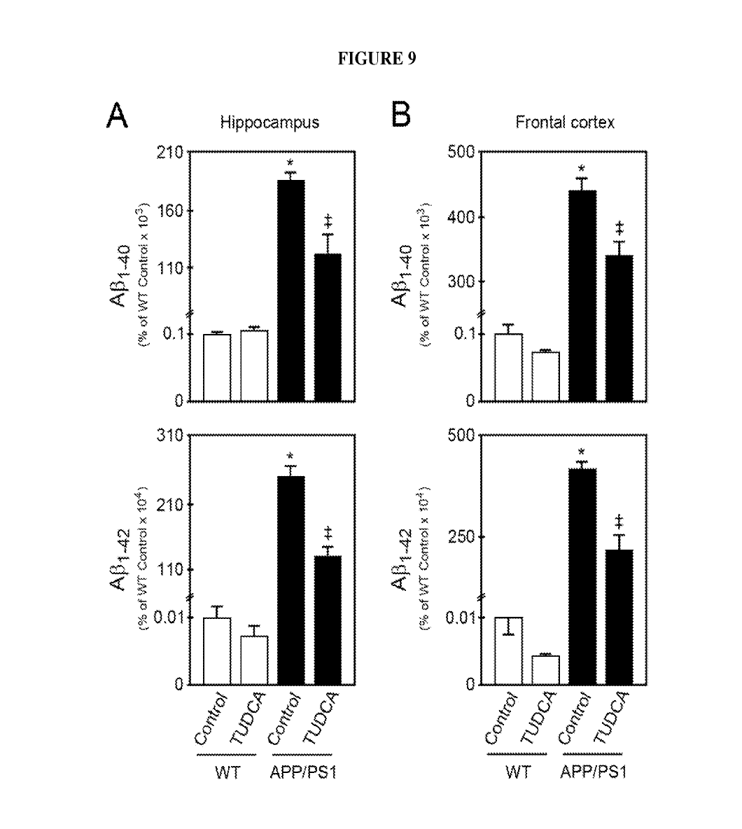

To further corroborate that TUDCA-mediated decrease in APP processing impacts on Aβ generation, we analyzed Aβ levels in the hippocampus and frontal cortex of untreated and TUDCA-treated wild-type and APP/PS1 brains by sandwich ELISA. Consistent with others, our results showed a dramatic increase in Aβ₁₋₄₀ and Aβ₁₋₄₂ levels in the brains of control APP/PS1 mice, with Aβ₁₋₄₂ exceeding Aβ₁₋₄₀ by ˜10-fold (FIGS. 9A and B). Aβ₁₋₄₂ to Aβ₁₋₄₀ ratio, known to be positively correlated with AD pathology was also strongly increased by ˜15-fold in control transgenic mice compared to wild-type mice (data not shown). Importantly, TUDCA treatment significantly decreased Aβ₁₋₄₀ and Aβ₁₋₄₂ levels in both hippocampus and frontal cortex of APP/PS1 mice (p<0.01) (FIGS. 9A and B), suggesting that TUDCA interferes with Aβ production, possibly through the regulation of lipid-metabolism mediators associated with APP processing.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

TABLE 1
Sense and antisense primers used to amplify
each cDNA of interest

| | Sense primer | Antisense |
|---|---|---|
| | (5′-3′) | primer (5′-3′) |
| Mouse | AGCCTCAAACTCCAAACACC | CAACAGGGATTTGACCAC |
| CTGF | | |
| Mouse | CTCAGCACCACAGAAACCAA | ATGAAGGAGGCACAGTGGAA |
| A2M | | |
| Mouse | TGTTTCGGAAGGAGCTGACT | TGTGTGACTTGGGAGCTCTG |
| APOE | | |
| Mouse | TAGCCTGGGAAGCCCAGC | TGGCAGCTTCCAGAGGTACAC |
| SORLA | | |
| Mouse | CATTGTGGAAGGGCTCATGAC | GCCCCACGGCCATCA |
| GAPDH | | |

1. A method of treating a neurodegenerative disease comprising administering an effective amount of a compound selected from the group consisting of a bile acid, a salt thereof, an analog thereof, and a combination thereof to a patient.

2. The method of claim 1, wherein the neurodegenerative disease is selected from the group consisting of Alzheimer's disease, Parkinson's disease, Huntington's disease and familial amyloidotic polyneuropathy.

3. The method of claim 1, wherein the compound administered to the nervous system is tauroursodeoxycholic acid (TUDCA).

4. The method of claim 3, wherein TUDCA administration inhibits a metabolic pathway leading to onset of the neurodegenerative disease.

5. The method of claim 3, wherein TUDCA administration activates a metabolic pathway which inhibits progression of the neurodegenerative disease.

6. The method of claim 1, wherein the compound administered to the nervous system comprises a TUDCA derivative, analog or precursor.

7. The method of claim 6, wherein the compound is selected from the group consisting of ursodeoxycholic acid (UDCA), a salt of TUDCA, a salt of UDCA and combinations thereof, wherein the salts are synthesized in a patient.

8. The method of claim 1, wherein the effective amount comprises between 1.0-60.0 mg/kg body weight.

9. The method of claim 8, wherein the effective amount comprises between 5.0-45.0 mg/kg body weight.

10. The method of claim 9, wherein the effective amount comprises between 15.0-40.0 mg/kg body weight.

11. The method of claim 10, wherein the effective amount comprises between 25.0-35.0 mg/kg body weight.

12. A method for treating a patient having a neurodegenerative disease, the method comprising administering to the patient an effective amount of a compound selected from the group consisting of TUDCA, UDCA, TUDCA salts, UDCA salts, TUDCA analogs, UDCA analogs and combinations thereof.

13. The method of claim 12, wherein the neurodegenerative disease is selected from the group consisting of Alzheimer's disease, Parkinson's disease, Huntington's disease and familial amyloidotic polyneuropathy.

14. The method of claim 12, wherein the compound is administered in combination with a pharmaceutical-grade carrier.

15. The method of claim 12, wherein the compound is administered orally, parenterally, intramuscularly, intravenously, or by direct administration into part of the brain.

16. The method of claim 12, wherein the compound is synthesized as a result of a therapy administered to the patient.

17. The method of claim 12, wherein the compound is synthesized as a result of a diet administered to the patient.

18. The method of claim 12, wherein an endogenous level of the compound is altered as a result of a therapy administered to the patient.

19. The method of claim 12, wherein an endogenous level of the compound is altered as a result of a diet administered to the patient.

20. The method of claim 12, wherein the compound is synthesized as a result of administration of foreign DNA into the patient.

21. A method of treating a neurodegenerative disease comprising administering a compound selected from the group consisting of TUDCA, UDCA, TUDCA salts, UDCA salts, TUDCA analogs, UDCA analogs and combinations thereof to a brain of a patient to diminish accumulation of amyloid-β (Aβ) peptide in the brain.

22. (canceled)

23. The method of claim 12, wherein the compound is unconjugated ursodeoxycholic acid.

24. (canceled)
